(19) United States
(12) Patent Application Publication
(10) Pub. No.: US 2017/0007356 A1
(43) Pub. Date: Jan. 12, 2017

(54) MANUAL ELECTROCAUTERY DEVICE

(71) Applicant: RAYMED, LLC, Wichita, KS (US)

(72) Inventor: Rachel Wykes, Wichita, KS (US)

(21) Appl. No.: 15/203,016

(22) Filed: Jul. 6, 2016

Related U.S. Application Data
(63) Continuation-in-part of application No. 14/792,812, filed on Jul. 7, 2015.

(51) Int. Cl. A61B 90/53; A61B 18/14

(52) U.S. Cl. CPC A61B 90/53; A61B 18/1402; A61B 2018/00589

(57) ABSTRACT

An electrocautery device includes two wearable cauterizers, each wearable cauterizer comprising a front bridge structure positioned above a knuckle on a top side of a hand, a back bridge structure positioned below the knuckle on the top side of the hand, at least one connecting member disposed between the front bridge structure and the back bridge structure, a cutting electrode disposed between the front bridge structure and the back bridge structure, and an insulator disposed between the cutting electrode and a finger, and a power unit.

### CROSS-REFERENCE TO RELATED APPLICATIONS

This application is a continuation-in-part of U.S. application Ser. No. 14/792,812 filed Jul. 7, 2015, the entire contents of which are incorporated herein by reference.

### TECHNICAL FIELD

Example embodiments relate generally to manual electrocautery devices for coagulation and cutting of tissue in a wide variety of surgical applications.

### BACKGROUND

Hysterectomies are the second most common surgical procedure in the U.S, with about 600,000 done annually. The four approaches for surgical removal of a uterus are abdominal, vaginal, laparoscopic, and robotic assisted laparoscopic. Objective evidence supports the total vaginal hysterectomy as the “gold standard” due to its low cost and safety. This approach demands technical skill and experience, which limits its application in many practices. The current instrumentation and approach for a vaginal hysterectomy includes metal clamps and suture ligation. The procedure requires the surgeon to manually identify four ligaments bilaterally to be clamped and sutured. The limitations include poor visualization in a small, cluttered working space. The easy part for the surgeon is to identify the basic anatomy with one's pointer and middle finger, enveloping the appropriate ligament and vasculature. Certain difficulty follows with tracing a Heney clamp along the fingers to grasp all of the selected tissue. The clamp's path is often obstructed with undesired vaginal soft tissue and the surgeon's gloves. This must be done to 4 ligaments bilaterally, in addition to extra vaginal tissue supporting the uterus. This is all done with little to no visualization. Once the clamp is placed, the surgeon must then confidently throw a stitch and tie knots to secure the vessels and tissue while simultaneously removing the clamp—again with very minimal visualization. The total number of knots tied by the surgeon can exceed 300 to secure this small space, which encompasses no more than 3-4 inches circumferentially. Inappropriate timing between removal of the clamp and placement of the stitch leads to devastating consequences including hemorrhage and distorted anatomy. The technical skill required limits its clinical application.

Robotic assist laparoscopic surgery has taken favor among gynecologists due to its technical ease and enhanced visualization and dexterity. Additionally the excitement for a new gadget has created popular demand among patients with disregard to best practice. This fad is not sustainable. A hospital must purchase a $2 million dollar apparatus, surgeons must enroll in hours of training, and an additional $2,200 is spent on each hysterectomy with no improvement in pain control, recovery time, or complications. There is a push among gynecologic surgeons and our pressured healthcare systems to re-embrace the gold standard of care for best practice and cost effectiveness and return to more vaginal surgery.

Therefore there remains a need in the art for a device designed to combine the assets of robotic surgery with the practicality of vaginal surgery to optimize the surgical experience for both patient and physician.

### BRIEF SUMMARY

One or more embodiments of the invention may address one or more of the aforementioned problems. Certain embodiments according to the present invention provide an electrocautery device suitable for a wide variety of applications. In accordance with certain embodiments, the electrocautery device may comprise two wearable cauterizers, each wearable cauterizer comprising a front bridge structure positioned above a knuckle on a top side of a hand, a back bridge structure positioned below the knuckle on the top side of the hand, at least one connecting member disposed between the front bridge structure and the back bridge structure, a cutting electrode disposed between the front bridge structure and the back bridge structure, and an insulator disposed between the cutting electrode and a finger. The electrocautery device may further comprise a power unit.

### BRIEF DESCRIPTION OF THE DRAWING(S)

The present invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout.

FIG. 1 illustrates a block diagram of an electrocautery device according to an example embodiment.

FIG. 2 illustrates a top view of two wearable cauterizers worn on a hand according to an example embodiment.

FIG. 3 illustrates a top view of a wearable cauterizer according to an example embodiment.

FIG. 4 illustrates a partial perspective view of an electrocautery device according to an example embodiment.

FIG. 5 a side view of a wearable cauterizer worn on a finger according to an example embodiment.

FIG. 6 illustrates a partial top view of an electrocautery device according to an example embodiment.

FIG. 7 illustrates a view of a wearable cauterizer according to an alternative example embodiment.

FIG. 8 shows a top view of a second wearable cauterizer according to an example embodiment.

FIG. 9 shows a top view of the first wearable cauterizer according to an example embodiment.

### DETAILED DESCRIPTION

Some example embodiments now will be described more fully hereinafter with reference to the accompanying drawings, in which some, but not all embodiments of the inventions are shown. Indeed, this invention may be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise.

An example embodiment includes an electrocautery device suitable for a wide variety of applications. In accordance with certain embodiments, the the electrocautery device may comprise two wearable cauterizers, each wearable cauterizer comprising a front bridge structure positioned above a knuckle on a top side of a hand, a back bridge structure positioned below the knuckle on the top side of the hand, at least one connecting member disposed between the front bridge structure and the back bridge structure, a cutting electrode (e.g., a wire, plate or other conductive surface) disposed between the front bridge structure and the back bridge structure, and an insulator disposed between the cutting electrode and a finger. The electrocautery device may further comprise a power unit.

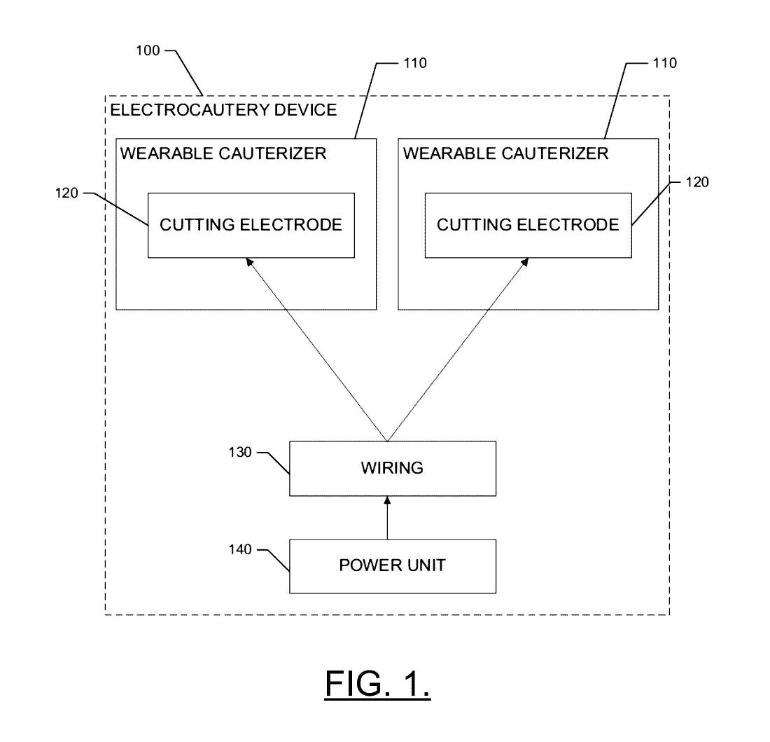

FIG. 1, for example, illustrates a block diagram of an electrocautery device according to an example embodiment. As shown in FIG. 1, the electrocautery device 100 includes two wearable cauterizers 110 having a cutting electrode 120 connected to a power unit 140 via wiring 130. The power unit 140 may be activated by a foot pedal or any other suitable activation means to enable the cutting electrode 120 to surgically coagulate, ligate, and/or cut tissue. The cutting electrode 120 may surgically coagulate, ligate, and/or cut tissue via bipolar cautery, ultrasonographic vibrations, or any other suitable means of coagulating, ligating, and/or cutting tissue.

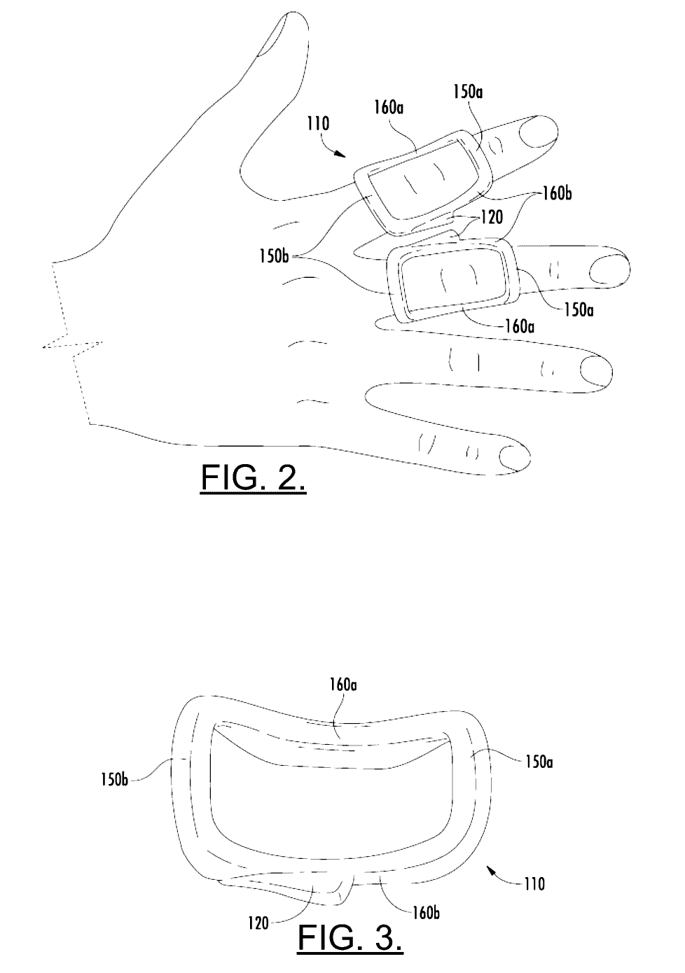

FIG. 2, for example, illustrates a top view of two wearable cauterizers worn on a hand according to an example embodiment. As shown in FIG. 2, each wearable cauterizer includes a cutting electrode 120, a front bridge structure 150a, a back bridge structure 150b, an external connecting member 160a, and an internal connecting member 160b. The wearable cauterizers 110 are worn on the index and middle finger of the hand. Each wearable cauterizer 110 is worn on a finger such that the external connecting member 160a and the internal connecting member 160b extend along opposite sides of the finger.

The wearable cauterizers 110 are positioned on the index and middle fingers respectively such that the cutting electrodes 120 of the wearable cauterizers 110 face each other in the space between the index finger and the middle finger. As such, an electrical current generated by the cutting electrodes 120 is passed between a surgeon's insulated hands. The surgeon's hand may be insulated via at least one of the connecting members 160a, 160b, the surgeon's glove, or a separate insulator. The insulator may selectively line the wiring 130 or may envelop the entire finger depending on the thermal spread generated by the cutting electrodes 120. Additionally, in some embodiments, the wearable cauterizers 100 may include a thermal spread sensor and/or a stopping mechanism to ensure surgeon safety when using the electrocautery device.

FIG. 3, for example, illustrates a top view of a wearable cauterizer according to an example embodiment. As shown in FIG. 3, the wearable cauterizer 110 includes a cutting electrode 120, a front bridge structure 150a, a back bridge structure 150b, an external connecting member 160a, and an internal connecting member 160b. In some embodiments, for example, the front bridge structure 150a and/or the back bridge structure 150b may be a plastic (e.g., lightweight plastic), metal, and/or fabric based on the surgical application for which the electrocautery device 100 will be used and the level of comfort required by the surgeon. If a fabric is used, the fabric may be a neoprene sleeve or any other fabric that has a high thermoinsulative capacity. The external connecting member 160a and/or the internal connecting member 160b may also be plastic, metal, fabric, rubber, or any other suitable material for connecting the front bridge structure 150a and the back bridge structure 150b. In certain embodiments, for instance, the internal connecting member 160b may be the insulator and, as such, is made of an insulating material. In other embodiments, for example, a connecting member may be the cutting electrode, as discussed below in further detail.

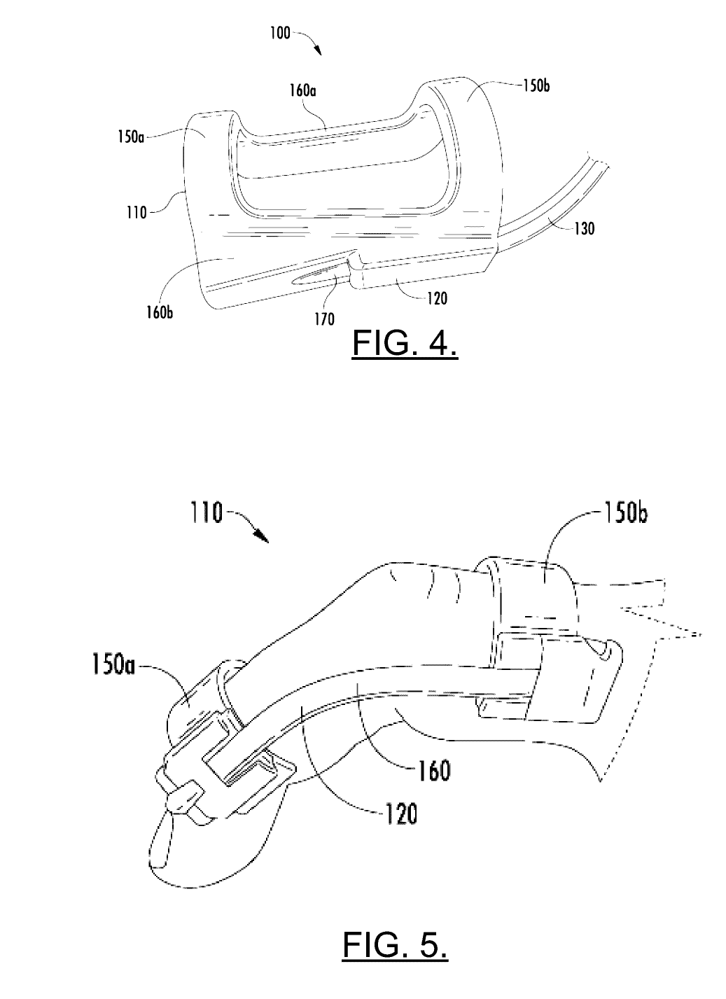

FIG. 4, for example, illustrates a partial perspective view of an electrocautery device according to an example embodiment. As shown in FIG. 4, the electrocautery device 100 includes two wearable cauterizers 110 (second wearable cauterizer not shown). The wearable cauterizer 110 includes a cutting electrode 120, a front bridge structure 150a, a back bridge structure 150b, an external connecting member 160a, an internal connecting member 160b, and a passthrough orifice 170. The passthrough orifice 170 extends through an insulator (the internal connecting member 160b in some embodiments) to the cutting electrode 120. As such, the wiring 130 may be built in to the electrocautery device 100, but a user's hand is protected from the wiring 130 due to the passthrough orifice 170 in the insulator. The wearable cauterizer 100 is connected to a power unit (not shown) via wiring 130. The wiring 130 may be secured within a surgeon's glove and gown and also may be grounded to the patient.

FIG. 5, for example, a side view of a wearable cauterizer worn on a finger according to an example embodiment. As shown in FIG. 5, the wearable cauterizer 110 includes a front bridge structure 150a, a back bridge structure 150b, a cutting electrode 120, and a connecting member 160, such that the cutting electrode 120 is the connecting member 160. The front bridge structure 150a and the back bridge structure 150b extend along both sides of a finger and terminate prior to the palm side of the finger. As such, the palm remains open while using the electrocautery device 100. Additionally, the front bridge structure 150a and the back bridge structure 150b are positioned on the finger so as to allow full joint motion.

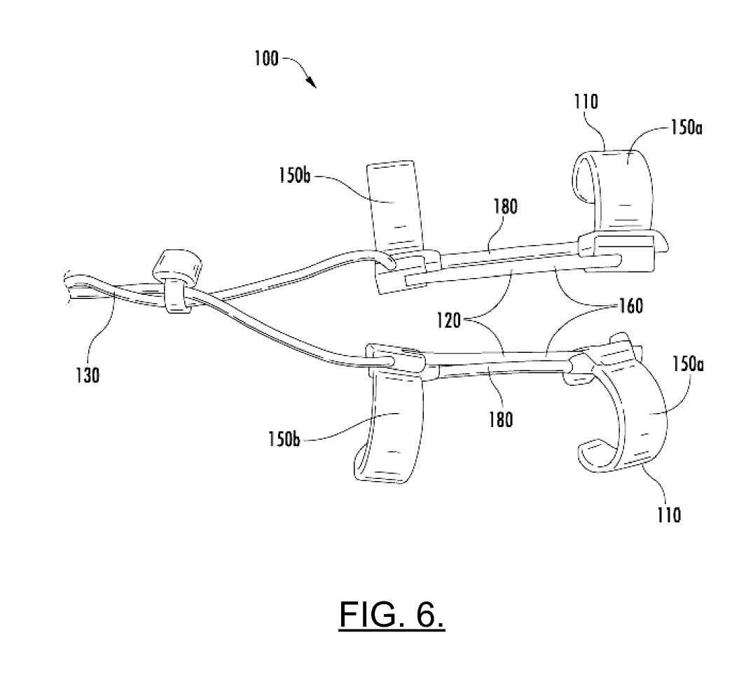

FIG. 6, for example, illustrates a partial top view of an electrocautery device according to an example embodiment. As shown in FIG. 6, the electrocautery device 100 includes two wearable cauterizers 110. Each wearable cauterizer 100 includes a front bridge structure 150a, a back bridge structure 150b, a rubber strip 180 (i.e. insulator), a cutting electrode 120, and a connecting member 160, such that the cutting electrode 120 is the connecting member 160. The two wearable cauterizers 110 are connected to a power unit (not shown) via wiring 130.

The rubber strip 180 may be replaced by another insulator including plastic, a glove, or any combination thereof. Alternatively, the insulator may be any other suitable insulating material. In FIG. 6, the rubber strip 180 extends between the front bridge structure 150a and the back bridge structure 150b. Additionally, the connecting member 160 includes the cutting electrode 120 and the rubber strip 180. (i.e. insulator). In some embodiments and as shown in FIG. 6, the cutting electrode 120 extends from the front bridge structure 150a to the back bridge structure 150b and may be entirely disposed between the front bridge structure 150a and the back bridge structure 150b. The cutting electrode 120 may be attached to the front bridge structure 150a and the back bridge structure 150b via a locking mechanism. The locking mechanism may be any suitable means of attaching the cutting electrode 120 to the front and back bridge structures 150a, 150b.

### Exemplary Embodiments

Having described various aspects and embodiments of the invention herein, further specific embodiments of the invention include those set forth in the following paragraphs.

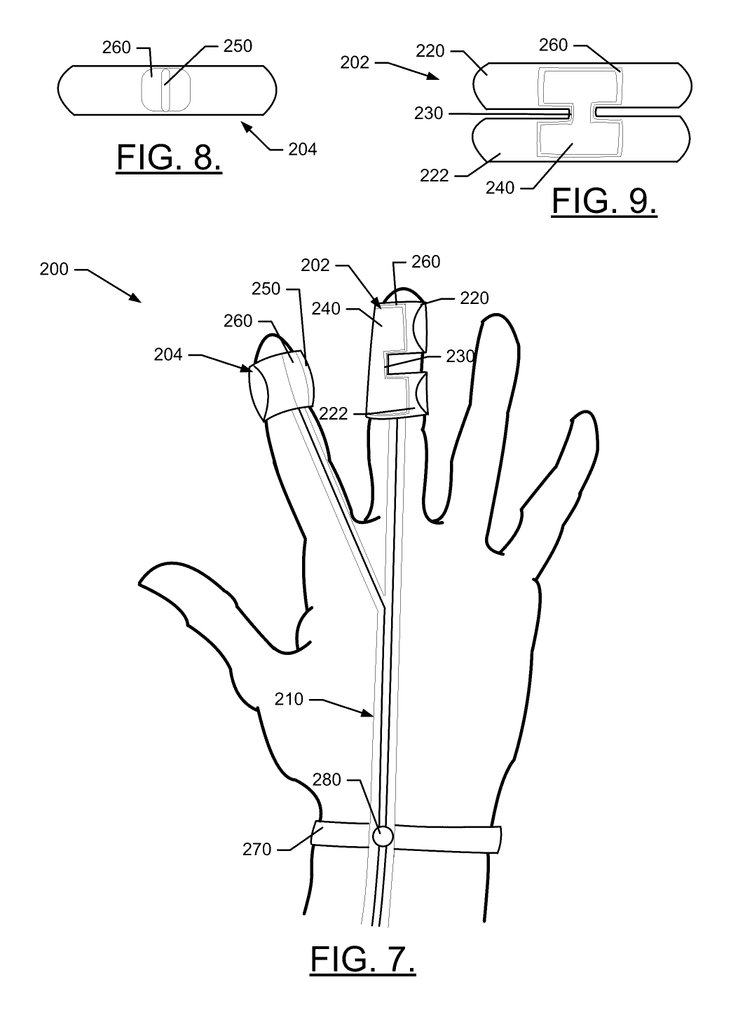

In some example embodiments, an electrocautery device is provided. In general, the electrocautery device, according to certain example embodiments, includes two wearable cauterizers and a power unit. In some cases, each wearable cauterizer comprises a front bridge structure positioned above a knuckle on a top side of a hand, a back bridge structure positioned below the knuckle on the top side of the hand, at least one connecting member disposed between the front bridge structure and the back bridge structure, a cutting electrode disposed between the front bridge structure and the back bridge structure, and an insulator disposed between the cutting electrode and a finger. However, in other example embodiments, only one wearable cauterizer may include both the front and rear bridge structures, and the other may include only one such bridge structure with an electrode attached thereto. FIG. 7 illustrates an alternative example embodiment that demonstrates this structure.

Referring now to FIG. 7, an electrocautery device 200 according to another example embodiment is provided. The electrocautery device includes two wearable cauterizers (e.g., first wearable cauterizer 202 and second wearable cauterizer 204) that are operably coupled to a power unit via cable 210. The second wearable cauterizer 204 of this example only includes a single bridge structure. However, the first wearable cauterizer 202 includes a front bridge structure 220 positioned above a knuckle on a top side of a hand, a back bridge structure 222 positioned below the knuckle on the top side of the hand. The knuckle in this case is the last knuckle of the index finger. However, another knuckle on this or another finger could be employed in other example embodiments.

In the example of FIG. 7, the front bridge structure 220 and back bridge structure 222 (and the bridge structure of the second wearable cauterizer 204) are each made of a cloth, fabric, plastic or other flexible material (e.g., rubber). Moreover, adhesive or a hook and loop fastener may be employed to enable the front bridge structure 220 and back bridge structure 222 to be affixed to the finger of the wearer. In this regard, the cloth, fabric, plastic or other flexible material may be wrapped around the finger and one end portion of the cloth, fabric, plastic or other flexible material may be attached to an opposing end portion of the cloth, fabric, plastic or other flexible material.

In the example of FIG. 7, a connecting member 230 may be disposed between the front bridge structure 220 and the back bridge structure 222 to connect the front bridge structure 220 to the back bridge structure 222. The connecting member 230 may be made from the same material that is used to form the front bridge structure 220 and the back bridge structure 222 and may connect adjacent middle portions of the front bridge structure 220 and the back bridge structure 222 to each other.

In this example, a cutting electrode in the form of a cutting pad 240 is disposed between the front bridge structure 220 and the back bridge structure 222. The cutting pad 240 also extends over significant portions the front bridge structure 220 and the back bridge structure 222 to form a butterfly shaped electrode. Meanwhile, a cutting electrode in the form of a cutting edge 250 may be formed by the electrode on the second wearable cauterizer 204. The cutting edge 250 may be shaped to have a narrow width and longer length extending along a longitudinal length of the finger on which the second wearable cauterizer 204 is worn. The cutting edge 250 may therefore extend transversely across the second wearable cauterizer 204 at or near a midpoint of the second wearable cauterizer 204.

The cutting edge 250 and the cutting pad 240 may each be substantially flat pieces of metallic or otherwise conductive material. However in some cases, at least the cutting edge 250 may be further shaped or sharpened to a point. In any case, an insulator 260 may be disposed between the cutting electrode and the finger on which its respective cauterizer is worn. Although the cloth, plastic or other flexible material itself could be the insulator 260 in some embodiments, other examples may provide the insulator 260 as a separate component extending around peripheral edges of the cutting edge 250 and the cutting pad 240 in all directions. By shaping the cutting edge 250 as a relatively longer and narrower (linear) component, but shaping the cutting pad 240 as a larger surface that is not necessarily linear, the area of intersection between the electrodes (i.e., the effective cutting surface) may typically be expected to be substantially linear, thereby defining a linear cutting pattern under most circumstances. If both cutting electrodes were linear, any misalignment of the linear cutting surfaces would result in an effective cutting surface that is very small and point-like instead of being linear.

As can be appreciated from the example of FIG. 7, the electrocautery device 200 can be worn on any hand and on any two fingers that the wearer chooses. Thus, the hand in FIG. 7 could be either the right hand or the left hand. However, some wearers may prefer to have the cable 210 extend over the back side of the hand. Regardless of which hand the electrocautery device 200 is used with, the cable 210 can be extended across the back (or front) of the hand. Furthermore, the cable 210 may also be secured to the wrist of the wearer (again) regardless of hand, or cable 210 routing) to prevent loose or dangling cable from becoming an issue.

In this regard, a wrist strap 270 may be worn by the user, and the cable 210 and wrist strap 270 may be joined together by a coupler 280. The coupler 280 of this example is a snap closure. Thus, the snap closure can be closed to attach the cable 210 to the wrist strap 270 in any arrangement simply by swapping the location and orientation of the portions of the snap closure to ensure that they align for the desired configuration. However, in alternative embodiments, in order to accommodate different sized hands, a hook and loop fastener may be employed as the coupler 280. For example, a piece or strip of hook material may be provided on the cable 210, and a piece or strip of loop material may be provided on the wrist strap 270. To ensure that any configuration can be supported and any length of hand, strips may be provided on one side of the wrist strap 270 (e.g., the outer side), but on both sides of the cable 210. Thus, either side of the cable 210 can be attached to the outer side of the wrist strap 270 so that a range of cable lengths can be defined between the wrist strap 270 and the first and second wearable cauterizers 202 and 204.

The cable 210 may also be configured to extend for at least a portion of its length along the hand with wires going to each respective cutting electrode being adjacent to (or even braided or wound with) each other. However, the cable 210 splits so that cable runs to each respective cutting electrode can extend along their corresponding fingers for maximum comfort with minimum excess cable. Typically, the electrocautery device 200 may be worn on adjacent fingers such that the cutting electrodes face each other. When worn in this way, the electrodes may face each other on adjacent portions of adjacent fingers, but the cable runs of the cable 210 may run along the back (or front) of the finger. Thus, the cable 210 may not align with the center of either of the cutting electrodes. Instead, the cable run of each respective portion of the cable 210 may intersect with the portions of the first wearable cauterizer 202 and the second wearable cauterizer 204 at a portion thereof at which one end of the insulator 260 begins.

FIG. 8 shows a top view of the second wearable cauterizer 204, and FIG. 9 shows a top view of the first wearable cauterizer 202, respectively. In both FIG. 8 and FIG. 9, the wearable cauterizers are simply unfolded from their respective arrangements in FIG. 7. Thus, as can be appreciated from FIGS. 7-9, the cable runs intersect with the opposite end portions of the insulator 260 of the first wearable cauterizer 202 and the second wearable cauterizer 204. In this regard, for example, the cable run to the cutting edge 250 intersects the second wearable cauterizer 204 at a left edge of the insulator 260, and the cable run to the cutting pad 240 intersects the first wearable cauterizer 202 at the right edge of the insulator 260. The cable runs may be defined as wires or other electrical conductors that are covered by an insulating materials (e.g., rubber or flexible plastic) to define the cable 210.

In accordance with certain example embodiments described above, a number of modifications, augmentations or optional additions to the general features described herein may be employed. For example, the front bridge structure and the back bridge structure may extend along both sides of the finger and terminate prior to a palm side of the finger. In some example embodiments, a first wearable cauterizer is disposed on an index finger and a second wearable cauterizer is disposed on a middle finger. In such embodiments, the cutting electrode of the first wearable cauterizer faces the cutting electrode of the second wearable cauterizer between the index finger and the middle finger.

In accordance with certain example embodiments, a foot pedal activates the power unit to enable the cutting electrode to surgically coagulate and cut tissue. In certain example embodiments, the cutting electrode surgically coagulates and cuts tissue via bipolar cautery. In other example embodiments, the cutting electrode surgically coagulates and cuts tissue via ultrasonographic vibrations.

In accordance with certain example embodiments, the insulator comprises at least one of a rubber strip, plastic, a glove, or any combination thereof. In certain exemplary embodiments, the insulator comprises a rubber strip extending between the front bridge structure and the back bridge structure. In some example embodiments, the at least one connecting member is the insulator.

In accordance with certain example embodiments, the front bridge structure and the back bridge structure comprise plastic. In other example embodiments, the front bridge structure and the back bridge structure comprise metal. In further example embodiments, the front bridge structure and the back bridge structure comprise fabric. In some example embodiments, the at least one connecting member comprises plastic.

In accordance with certain example embodiments, each wearable cauterizer comprises one connecting member. In such embodiments, the connecting member comprises the cutting electrode and a rubber strip disposed between the cutting electrode and the finger. In some example embodiments having one connecting member, the cutting electrode extends from the front bridge structure to the back bridge structure. In certain example embodiments, the cutting electrode is entirely disposed between the front bridge structure and the back bridge structure.

In other example embodiments, each wearable cauterizer comprises two connecting members. In such embodiments, each wearable cauterizer comprises an internal connecting member and an external connecting member, and the internal connecting member and the external connecting member extend along opposite sides of the finger. In some example embodiments, each wearable cauterizer can be modified to further comprise a passthrough orifice extending through the insulator to the cutting electrode.

These and other modifications and variations to the present invention may be practiced by those of ordinary skill in the art without departing from the spirit and scope of the present invention, which is more particularly set forth in the appended claims. In addition, it should be understood that aspects of the various embodiments may be interchanged in whole or in part. Furthermore, those of ordinary skill in the art will appreciate that the foregoing description is by way of example only, and it is not intended to limit the invention as further described in such appended claims. Therefore, the spirit and scope of the appended claims should not be limited to the exemplary description of the versions contained herein.

That which is claimed:

1. An electrocautery device, comprising:
two wearable cauterizers, at least one of the wearable cauterizers comprising a front bridge structure positioned above a knuckle on a top side of a hand, a back bridge structure positioned below the knuckle on the top side of the hand, at least one connecting member disposed between the front bridge structure and the back bridge structure, a cutting electrode disposed between the front bridge structure and the back bridge structure, and an insulator disposed between the cutting electrode and a finger; and
a power unit.

2. The electrocautery device according to claim 1, wherein the front bridge structure and the back bridge structure extend along both sides of the finger.

3. The electrocautery device according to claim 1, wherein a first wearable cauterizer is disposed on an index finger and a second wearable cauterizer is disposed on a middle finger, wherein the cutting electrode of the first wearable cauterizer faces the cutting electrode of the second wearable cauterizer between the index finger and the middle finger.

4. The electrocautery device according to claim 1, wherein a foot pedal activates the power unit to enable the cutting electrode to surgically coagulate and cut tissue.

5. The electrocautery device according to claim 1, wherein the cutting electrode surgically coagulates and cuts tissue via bipolar cautery or via ultrasonographic vibrations.

6. The electrocautery device according to claim 1, wherein the cutting electrode has a butterfly shape extending across both the front bridge structure and the back bridge structure, and wherein the other of the two wearable cauterizers comprises a single bridge structure and a substantially linear shaped cutting electrode that extends substantially perpendicular to a longitudinal centerline of the other of the two wearable cauterizers.

7. The electrocautery device according to claim 1, wherein the insulator comprises at least one of a rubber strip, plastic, a glove, or any combination thereof.

8. The electrocautery device according to claim 1, wherein the insulator comprises a rubber strip extending between the front bridge structure and the back bridge structure.

9. The electrocautery device according to claim 1, wherein the at least one connecting member is the insulator.

10. The electrocautery device according to claim 1, wherein the front bridge structure and the back bridge structure comprise plastic, metal or fabric.

11. The electrocautery device according to claim 1, wherein the front bridge structure and the back bridge structure comprise a flexible material and an adhesive used to attach the at least one of the wearable cauterizers to the finger.

12. The electrocautery device according to claim 1, wherein only one of the wearable cauterizers includes both the front bridge structure and the back bridge structure, and the other wearable cauterizer includes only a single bridge structure and a second cutting electrode extending therefrom.

13. The electrocautery device according to claim 12, wherein each of the two wearable cauterizers is operably coupled to the power unit via a cable.

14. The electrocautery device according to claim 13, further comprising a wrist strap, the wrist strap being operably coupled to the cable via a coupler.

15. The electrocautery device according to claim 14, wherein the coupler comprises a snap closure or a hook and loop fastener.

16. The electrocautery device according to claim 14, wherein the coupler is configured to enable either side of the cable to be attached to an outer side of the wrist strap to define a range of cable lengths achievable between the wrist strap and the two wearable cauterizers.

17. The electrocautery device according to claim 14, wherein the second cutting electrode has a linear shape and the cutting electrode of the at least one of the wearable cauterizers forms a cutting pad having a non-linear shape.

18. The electrocautery device according to claim 16, wherein each of the second cutting electrode and the cutting electrode is adjacent to a respective instance of the insulator along an entirety of the second cutting electrode and the electrode.

19. The electrocautery device according to claim 17, wherein the cutting electrode and the second cutting electrode face each other on adjacent portions of adjacent fingers, and cable runs of the cable extend along a back portion of each of the adjacent fingers.

20. The electrocautery device according to claim 18, wherein the cable runs of the cable intersect opposite ends of the respective instances of the insulator.
